(19) United States
(12) Patent Application Publication
(10) Pub. No.: US 2025/0213348 A1
(43) Pub. Date: Jul. 3, 2025

(54) TUBE & CABLE ACTUATED ENDOSCOPIC GRAFT INSERTER

(71) Applicant: Zimmer, Inc., Warsaw, IN (US)

(72) Inventor: Donald W. Dye, JR., Warsaw, IN (US)

(21) Appl. No.: 18/990,436

(22) Filed: Dec. 20, 2024

Related U.S. Application Data
(60) Provisional application No. 63/615,628, filed on Dec. 28, 2023.

(51) Int. Cl. A61F 2/08

(52) U.S. Cl. CPC A61F 2/0805

(57) ABSTRACT

An implant delivery device can include a handle disposed at a proximal end of the implant delivery device and configured to be grasped by a user, a first outer bendable shaft extending from the handle to a distal end of the implant delivery device and a second outer bendable shaft extending from the handle to a distal end of the implant delivery device and positioned adjacent to the first outer bendable shaft, and an implant holding portion proximate the distal end of the first and second outer bendable shafts, the implant holding portion being configured to retain a sheet-like implant during implantation of the sheet-like implant by being attached to outer edges of the sheet-like implant, wherein a distal end of each of the first and second outer bendable shafts is configured to bend to convert the sheet-like implant from a collapsed delivery configuration to a spread-out deployment configuration.

### CLAIM OF PRIORITY

This application claims the benefit of U.S. Provisional Patent Application Ser. No. 63/615,628, filed on Dec. 28, 2023, the benefit of priority of which is claimed hereby, and which is incorporated by reference herein in its entirety.

### TECHNICAL FIELD

The present embodiments relate generally to medical devices, and in particular to medical devices used to repair tissue.

### BACKGROUND

To repair tendons, ligaments, muscles, rotator cuffs, and other soft tissue, a graft implant can be placed and implanted to fix the body part. For example, rotator cuff repair is a surgical procedure performed to repair torn (or partially torn) tendons in the shoulder. This procedure can be done with large incisions or with arthroscopic techniques. To repair a torn tendon (such as the supraspinatus tendon), a surgeon may use anchors and sutures to reattach the tendon to the humerus bone. The repaired area may then be covered with a graft to facilitate healing. Inserting a graft through a small incision and laying it down in a desired position can be difficult with arthroscopic surgery.

There is a need in the art for a system and method that addresses the shortcomings discussed above.

### OVERVIEW

To further illustrate apparatuses and methods disclosed herein, a non-limiting list of examples is provided here:

Example 1 is an implant delivery device including a handle disposed at a proximal end of the implant delivery device and configured to be grasped by a user, a first outer bendable shaft extending from the handle to a distal end of the implant delivery device and a second outer bendable shaft extending from the handle to a distal end of the implant delivery device and positioned adjacent to the first outer bendable shaft, and an implant holding portion proximate the distal end of the first and second outer bendable shafts, the implant holding portion being configured to retain a sheet-like implant during implantation of the sheet-like implant by being attached to outer edges of the sheet-like implant, wherein a distal end of each of the first and second outer bendable shafts is configured to bend to convert the sheet-like implant from a collapsed delivery configuration to a spread-out deployment configuration.

Example 2 is the implant delivery device of Example 1, wherein optionally the distal end of the first outer bendable shaft bends outwards from a longitudinal axis in one direction and the distal end of the second outer bendable shaft bends outwards from a longitudinal axis in a second, opposite direction.

Example 3 is the implant delivery device of any of examples 1-2, wherein optionally the implant holding portion includes a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant, and wherein as the distal ends of the first and second outer bendable shafts bend outwards, the sheet-like implant becomes stretched between the first inner implant holding rod and the second inner implant holding rod.

Example 4 is the implant delivery device of any of Example 1-3, wherein optionally the distal end of each of the first and second outer bendable shafts includes a plurality of notches configured to bias the first and second bendable shafts to bend when a force is exerted on each end of the first and second bendable shafts.

Example 5 is the implant delivery device of Example 4, wherein optionally the plurality of notches on each of the first and second outer bendable shafts includes a group of proximally located notches on an outer surface of each of the first and second outer bendable shafts and a group of distally located notches on an inner surface of each of the first and second outer bendable shafts.

Example 6 is the implant delivery device of any of Examples 4-5, wherein optionally the implant holding portion includes a first inner implant holding rod located within the first outer bendable shaft and a second inner implant holding rod located within the second outer bendable shaft, and wherein the force is exerted on a proximal end of each of the first and second outer bendable shafts by an actuator within the handle and an opposite force is exerted on a distal end of each of the first and second outer bendable shafts by a collar on each of the first and second inner implant holding rods.

Example 7 is the implant delivery device of Example 6, wherein optionally when the force is removed on each end of the first and second bendable shafts, the first and second bendable shafts straighten out.

Example 8 is the implant delivery device of any of Examples 1-7, wherein optionally the implant holding portion includes a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant, and wherein the sheet-like implant includes outer loops at each side to connect with the first and second inner implant holding rods, and after being implanted, the first and second inner implant holding rods are retracted through the loops, leaving the sheet-like implant in place.

Example 9 is the implant delivery device of any of Examples 1-7, wherein optionally the implant holding portion includes a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant, and wherein the sheet-like implant is configured such that, after being implanted, the first and second outer bendable shafts and first and second inner implant holding rods are retracted and sacrificial portions of the sheet-like implant are torn way by the first and second inner implant holding rods, leaving the sheet-like implant in place.

Example 10 is the implant delivery device of Example 9, wherein optionally the sacrificial portions of the sheet-like implant include tear-away portions of outer hemmed loops on each side of the sheet-like implant.

Example 11 is the implant delivery device of Example 10, wherein optionally each of the first and second inner implant holding rods includes an expanded tip portion to engage the outer hemmed loops as the first and second inner implant holding rods are retracted so as to tear away portions of the hemmed loops.

Example 12 is the implant delivery device of Example 11, wherein optionally the expanded tip portion of each of the first and second inner implant holding rods includes a half-dome member, and the half-dome members mate to form a full-dome shape when the first and second inner implant holding rods are together.

Example 13 is the implant delivery device of any of Example 9, wherein optionally the sacrificial portions of the sheet-like implant includes tear away eyelets at each corner of the sheet-like implant.

Example 14 is the implant delivery device of any of Example 9, wherein optionally the sacrificial portions of the sheet-like implant includes a hole located at each corner of the sheet-like implant and the first and second inner implant holding rods each include a pair of hooks to engage the sheet-like implant at each of the holes.

Example 15 is an implant delivery device including an implant holding portion proximate a distal end of the implant delivery device, the implant holding portion being configured to retain a sheet-like implant during implantation of the sheet-like implant, wherein the implant holding portion is configured to hold the sheet-like implant in a collapsed delivery configuration while the sheet-like implant is pushed through a cannula and to stretch the sheet-like implant at its outer edges until the sheet-like implant is in a spread-out deployment configuration.

Example 16 is the implant delivery device of Examples 15, optionally further including first and second outer bendable shaft which are configured to bend with the distal end of the first outer bendable shaft bending in one direction and the distal end of the second outer bendable shaft being bent in a second, opposite direction, wherein as the distal ends of the first and second outer bendable shafts are bent, the sheet-like implant becomes stretched, and wherein the implant holding portion includes a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant.

Example 17 is the implant delivery device of any of Examples 15-16, wherein optionally the distal end of each of the first and second outer bendable shafts includes a plurality of notches configured to bias the first and second bendable shafts to bend when a force is exerted on each end of the first and second bendable shafts.

Example 18 is a method for implanting a sheet-like implant including inserting a sheet-like implant through a cannula while the sheet-like implant is in a collapsed delivery configuration, and stretching the sheet-like implant out by applying stretching forces at outer edges of the sheet-like implant until the sheet-like implant is in a spread-out deployment configuration.

Example 19 is the method of Example 18, wherein stretching the sheet-like implant out includes attaching one side of the sheet-like implant to a first inner implant holding rod and attaching a second side of the sheet-like implant to a second inner implant holding rod; and bending the first and second inner implant holding rods away from each other.

Example 20 is the method of any of Example 19, wherein bending the first and second inner implant holding rods away from each other includes mounting each of the first and second inner implant holding rods within a first outer bendable shaft and a second outer bendable shaft, respectively, wherein a distal end of each of the first and second outer bendable shaft are configured to bend to convert the sheet-like implant from the collapsed delivery configuration to the spread-out deployment configuration.

In Example 21, the apparatuses or methods of any one or any combination of Examples 1-20 can optionally be configured such that all elements or combinations of elements or options recited are available to use or select from.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatus, systems, and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic view of a step in a procedure for repairing a rotator cuff tendon according to an embodiment.

FIG. 2 is a schematic view of a step of applying an implant to a portion of a rotator cuff tendon to facilitate healing according to an embodiment.

FIG. 3 is a schematic illustration of an implant delivery device in accordance with one embodiment.

FIG. 4 shows a top perspective view of an implant, in accordance with one embodiment.

FIG. 5 shows a top view of the implant mounted to the delivery device, in accordance with one embodiment.

FIG. 6 shows a top view of the implant as deployed, in accordance with one embodiment.

FIG. 7 shows a side view of a portion of a distal end of the delivery device, in accordance with one embodiment.

FIG. 8 shows another side view of a portion of a distal end of the delivery device, in accordance with one embodiment.

FIG. 9 shows the implant and the implant delivery device, in accordance with one embodiment.

FIG. 10 shows a top view of an implant, in accordance with one embodiment.

FIG. 11 shows a side view of the implant, in accordance with one embodiment.

FIG. 12 shows a perspective view of an implant and delivery device, in accordance with one embodiment.

FIG. 13 shows a perspective view of the implant and delivery device, in accordance with one embodiment.

FIG. 14 shows a perspective view of the implant and delivery device, in accordance with one embodiment.

FIG. 15 shows a perspective view of an implanted implant, in accordance with one embodiment.

FIG. 16 shows a top view of an implant, in accordance with one embodiment.

FIG. 17 shows a top view of an implant and a delivery device, in accordance with one embodiment.

FIG. 18 shows a top view of an implant, in accordance with one embodiment.

FIG. 19 shows a method for implanting an implant, in accordance with one embodiment.

### DETAILED DESCRIPTION

For clarity, the description makes reference to distal and proximal directions (or portions). As used herein, the term “distal” shall refer to a direction or portion oriented or located away from a user who is holding the implant delivery device (i.e., away from a surgeon using the device and toward a patient into which the device is being inserted). The term “proximal” shall refer to a direction or portion oriented or located toward a user who is holding the implant delivery device (i.e., toward a surgeon using the device and away from a patient into which the device is being inserted).

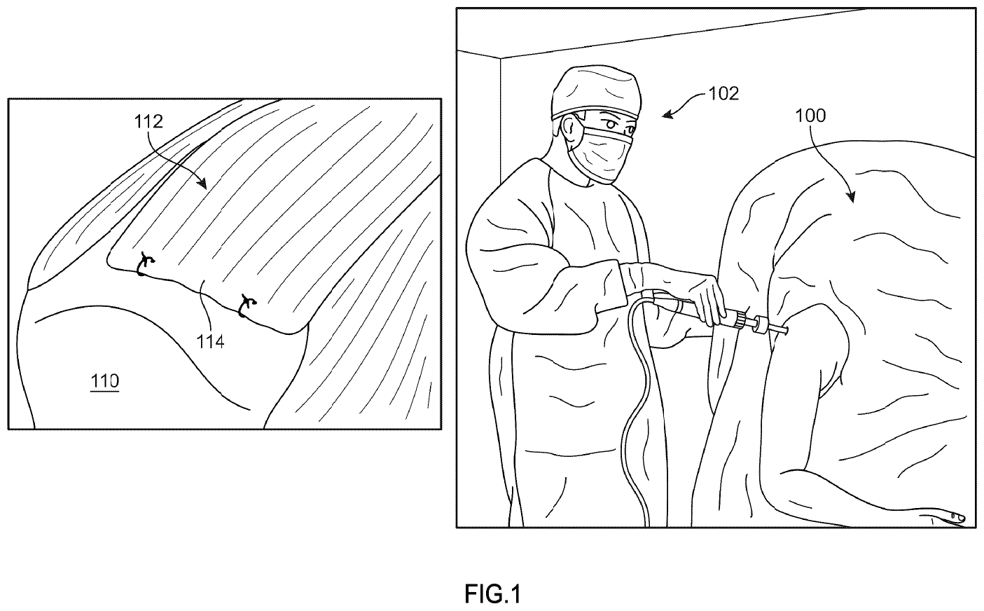

FIG. 1 is a schematic view illustrating a surgical procedure to repair a tendon in a patient's shoulder. Specifically, a patient 100 is undergoing arthroscopic surgery that is performed by surgeon 102. Also shown in FIG. 1 is an enlarged view of a portion of humerus 110 and rotator cuff tendons 112. In the present example, surgeon 102 has recently applied anchors and sutures to secure supraspinatus tendon 114 to humerus 110.

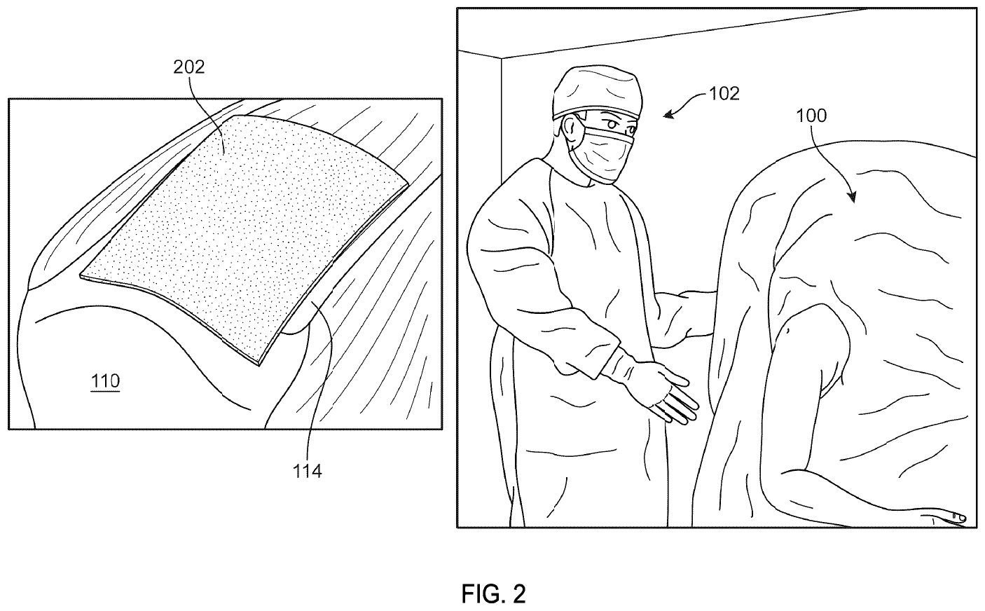

Once the tendon has been sufficiently repaired and/or the surgical site is otherwise prepared, surgeon 102 may insert an implant, such as a graft, through an incision (possibly using another device to facilitate insertion). The graft can then be placed over the tendon and/or portion of the underlying bone in order to facilitate healing. As an example, FIG. 2 shows a schematic view of a graft 202 that has been applied over the tendon 114 as well as over a portion of humerus 110.

Although the exemplary embodiment depicts a procedure in which a tendon is first secured to the bone using sutures and anchors, in other embodiments a graft can be applied to one or more tendons without first reattaching a tendon. For example, grafts could be applied to tendons that have only partial tears.

Once the implant or graft 202 has been placed over the tendon, one or more sutures or anchors are required to hold graft 202 in place. The present embodiments disclose a graft delivery device configured to hold an implant, such as a graft, during insertion and release the implant/graft once it has been secured at the surgical site.

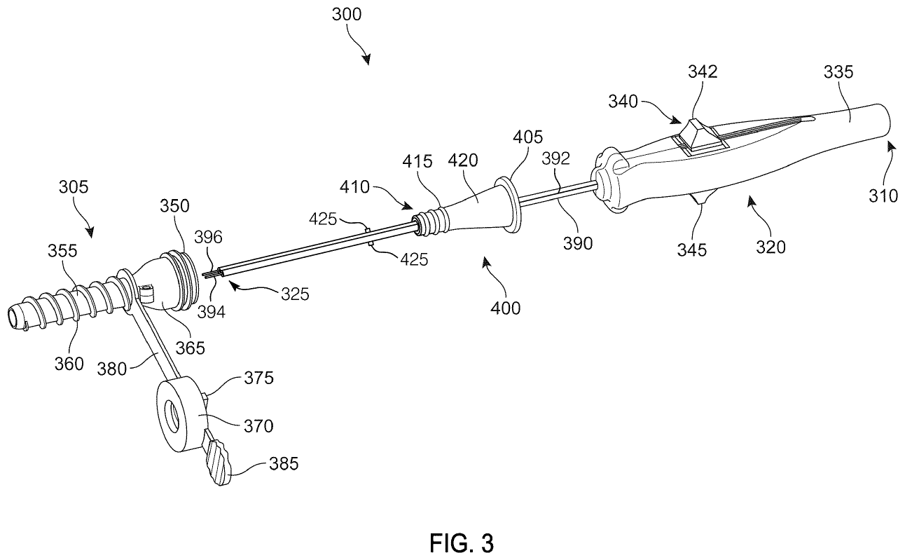

As noted above, inserting an implant graft through a small incision and laying it down in a desired position can be difficult during surgery. Examples of an implant delivery device that can be used to insert an implant through a small incision and position it in over tissue in a fully opened condition is shown in various embodiments in FIGS. 3-18.

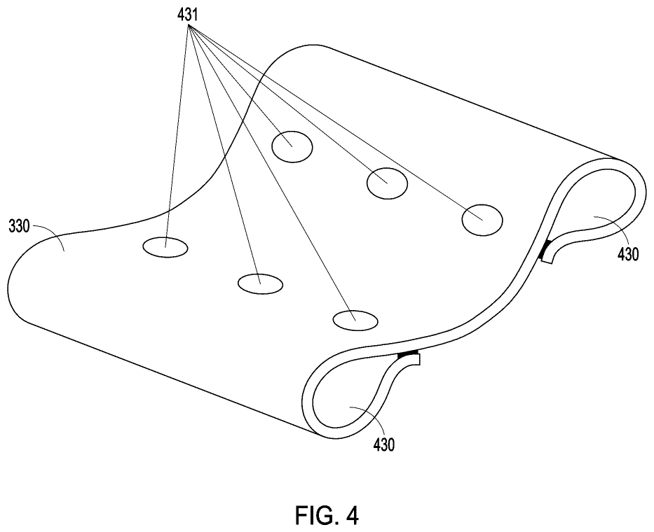

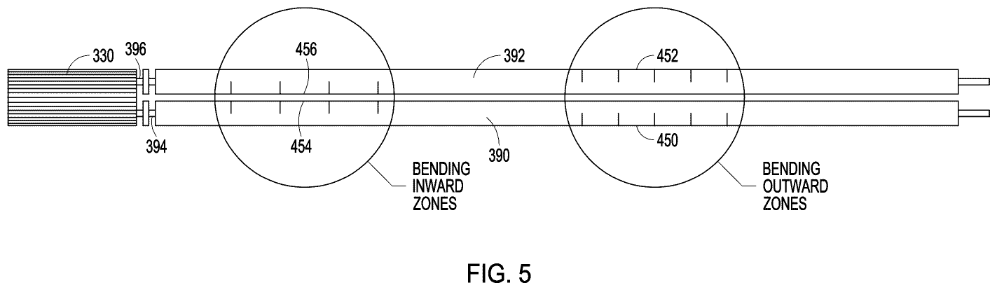

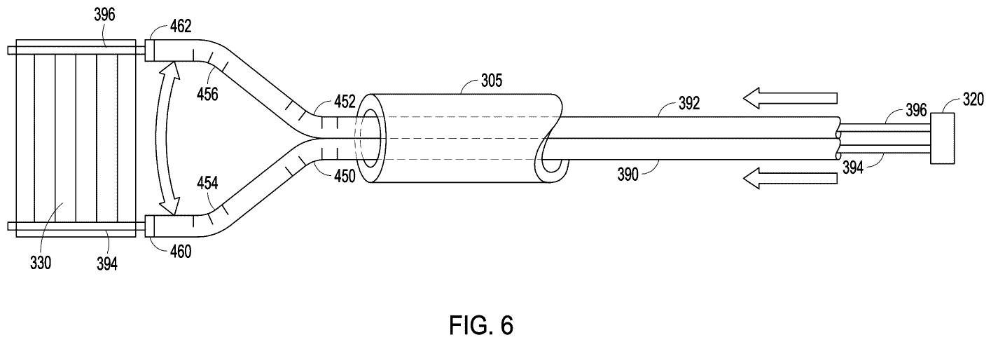

FIG. 3 is a schematic illustration of an implant delivery device 300 according to an embodiment; FIG. 4 shows a top perspective view of an implant 330, in accordance with one embodiment; FIG. 5 shows a top view of the implant 330 mounted to the delivery device 300 before deployment; and FIG. 6 shows a top view of the implant 330 as deployed, in accordance with one embodiment.

In one embodiment, the implant delivery device 300 may have a proximal end 310 and a distal end 315. The implant delivery device 300 can generally include a handle 320 disposed at the proximal end 310 of the implant delivery device 300 and configured to be grasped by a user. The device further includes a first outer bendable shaft 390 extending from the handle 320 to a distal end of the implant delivery device 300 and a second outer bendable shaft 392 extending from the handle 320 to a distal end of the implant delivery device 300 and positioned adjacent to the first outer shaft 390. Accordingly, the first and second outer bendable shafts 390, 392 run parallel to each other along their lengths.

Implant delivery device 300 may include an implant holding portion 325 proximate distal end 315 of implant delivery device 300, implant holding portion 325 being configured to retain a sheet-like implant 330 during implantation of implant 330.

In some embodiments, the implant 330 may be formed, at least in part of collagen. In other embodiments, the sheet-like implant may be formed of synthetic material or blends of collagen and various synthetic polymers.

The handle 320 may include a casing 335 including one or more slider members or actuators 340, 345 configured to actuate bending mechanism discussed in greater detail below. The casing 335 of handle 320 may be formed of a substantially rigid material, such as a hard/rigid plastic and/or metal. Casing 335 must substantially maintain its shape and structural integrity during manipulation of implant delivery device 300 as handle 320 is the portion of the device to which forces are applied during use.

As shown in FIG. 3, a cannula 305 may have a proximal end 350 with a wider mouth opening and a narrower distal portion 355 including threads 360. Cannula 305 may include a tapered intermediate portion 365 between proximal end 350 and distal portion 355. As further shown in FIG. 3, cannula 305 may include a seal structure 370. Seal structure may include a seal 375. Seal 375 may be any type of seal suitable for passing instruments, such as obturators through. For example, as shown in the accompanying figures, seal 375 may be a duckbill valve. In other embodiments, seal 375 may be a tricuspid valve. In some embodiments, seal structure 370 may include more than one seal in series. In addition, seal structure 370 may include a tether 380 for connecting seal structure 370 to cannula 305. Further, seal structure 370 may include a grasping tab 385. It will be understood that cannula 305 and seal structure 370 may have any suitable configurations for passing implants therethrough using implant delivery device 300.

The implant delivery device 300 may further include a cannula sealing member 400 disposed about a midportion of outer shafts 390, 392. Cannula sealing member 400 may include a proximal flange 405 for abutting proximal end 350 of cannula 305. In addition, cannula sealing member 400 may include a narrower distal portion 410 configured to fit within narrow distal portion 355 of cannula 305. Distal portion 410 of cannula sealing member 400 may include one or more ribs 415 configured to seal against the inner wall of distal portion 355 of cannula 305. In addition, cannula sealing member 400 may include a tapered intermediate portion 420 between proximal flange 405 and narrow distal portion 410.

When implant insertion device 300 is to be inserted through cannula 305, seal structure 370 may be removed from proximal end 350 of cannula 305. In order to maintain pressure at the surgical site, cannula sealing member 400 may be inserted into cannula 305 as implant delivery device 300 is passed through cannula 305. Cannula sealing member 400 may be slidable along outer shafts 390, 392 of implant insertion device 300 in order to facilitate manipulation of implant insertion device 300 to and about the surgical site.

The bendable outer shafts 390, 392 of implant delivery device 300 may include one or more stop portions 425 disposed distal to cannula sealing member 400 and configured to pull cannula sealing member 400 out of cannula 305 when handle 320 of implant delivery device 300 is pulled in the proximal direction to withdraw implant delivery device 300 from the surgical site.

As noted, the implant delivery device 300 includes the implant holding portion 325 proximate the distal end of the implant delivery device 300. The implant holding portion 235 is configured to retain a sheet-like graft or implant 330 during implantation of the sheet-like implant 330. In this example, the implant holding portion 325 includes a first inner implant holding rod 394 located within the first outer bendable shaft 390 and a second inner implant holding rod 396 located within the second outer bendable shaft 392.

Implant grafts can be fragile and hard to handle when implanting. For example, the edges and corners of the implant are prone to flapping when fluid flushing through an endoscopic passes past the implant. For example, many arthroscopic surgeries, such as joint surgeries, use pressurized irrigation fluid to keep tissue separated apart from other tissue. In particular, pressurized irrigation fluid may be used to aid in visualization of the surgical site as well as to prevent bleeding from vasculature surrounding the surgical cuts.

Accordingly, the present implant delivery device 300 securely captures all four corners of the fragile implant graft and, when actuated, stretches the implant's width to its maximum dimension and holds it there until the implant is stapled, or otherwise secured, in place in a patient.

As shown in FIG. 5, the first inner implant holding rod 394 is configured to be removably attached to one side of the sheet-like implant 330 and the second inner implant holding rod 396 is configured to be removably attached to a second side of the sheet-like implant 330. A distal end of each of the first and second outer bendable shafts 390, 392 are configured to bend so as to bend the first and second inner implant holding rods 394, 396 to convert the sheet-like implant from a collapsed delivery configuration to a spread-out deployment configuration.

FIG. 6 shows how the distal end of the first outer bendable shaft 390 bends outwards from a longitudinal axis in one direction and the distal end of the second outer bendable shaft 392 bends outwards from a longitudinal axis in a second, opposite direction. As the distal ends of the first and second outer bendable shafts 390, 392 bend outwards, the sheet-like implant 330 becomes stretched between the first inner implant holding rod 394 and the second inner implant holding rod 396.

The distal end of each of the first and second outer bendable shafts 390, 392 can include a plurality of notches 450, 452, 454, 456 configured to bias the first and second bendable shafts 390, 392 to bend when a force is exerted on each end of the first and second bendable shafts 390, 392.

The plurality of notches 450, 452, 454, 456 on each of the first and second outer bendable shafts 390, 392 can include a group of proximally located notches 450, 452 on an outer surface of each of the first and second outer bendable shafts 390, 392 and a group of distally located notches 454, 456 on an inner surface of each of the first and second outer bendable shafts 390, 392.

The first and second outer bendable shafts 390, 392 will bend when a force is exerted on a proximal end of each of the first and second outer bendable shafts 390, 392 by an actuator within the handle, and an opposite force is exerted on a distal end of each of the first and second outer bendable shafts by a collar 460, 462 on each of the first and second inner implant holding rods 394, 396.

In one example, each of the inner rods 394, 396 can be solidly attached with the handle 320. Accordingly, the outer bendable shafts 390, 392 can slide over the inner rods 394, 396. When the actuator 342, 345 is actuated, the actuator applies a pushing force on the proximal ends of the outer bendable shafts 390, 392 forcing the outer bendable shafts 390, 392 to slide over the inner rods 394, 396 until a distal end of each of the outer shafts 390, 392 strikes the collars 460, 462. When this happens, the notches 450, 452, 454, 456 bias the outer shafts into the curved, deployment position as shown in FIG. 6. It is noted that the inner rods 394, 396 are flexible and are bent by the outer shafts 390, 392 as the outer shafts are bent.

In another example, the outer bendable shafts 390, 392 can be solidly attached to the handle 320 and the inner rods 394, 396 can be movable and can actuated by pulling the rods 394, 396 towards the handle using the actuator 342, 345. When the collars 460, 462 push against the distal ends of the outer shafts 390, 392, the shafts will bend due to the notches 450, 452, 454, 456. This results in a rigid construct as long as tension is maintained. When the tension on the inner rods 394, 396 is released, the outer shafts 390, 392 resume their ‘straight line’ configuration and the inserter can be removed through the same cannula 305 it was introduced with. In an example, using either actuating technique the outer shafts 390, 392 can be bent by being actuated together using one actuator 342, 345, or separately using separate actuators.

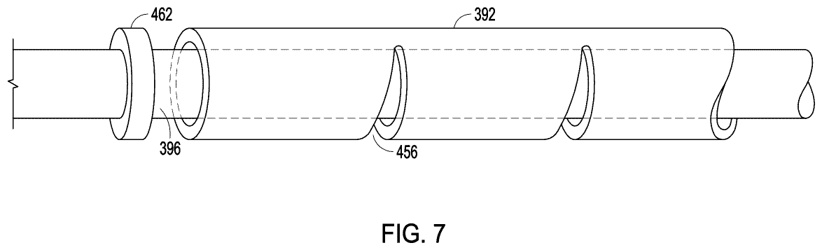

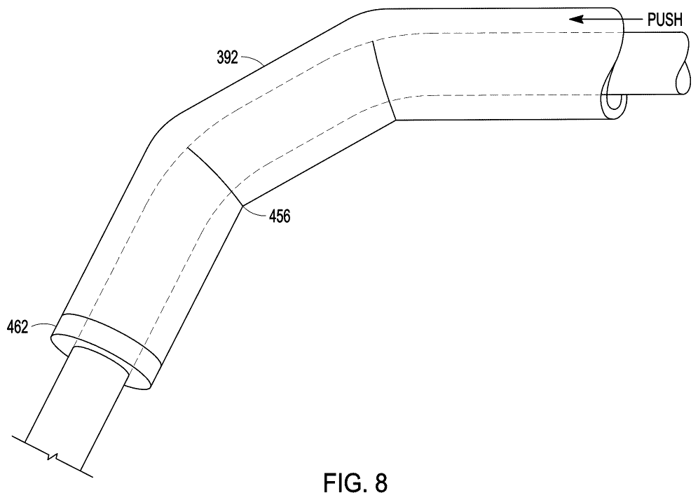

FIG. 7 shows a side view of a portion of a distal end of the delivery device, in accordance with one embodiment; and FIG. 8 shows another side view of a portion of a distal end of the delivery device. FIGS. 7, 8 show further details of how the notches 456 can be formed in the outer bendable shafts 390, 392 and how when a force is applied at the proximal end of the outer bendable shaft 392 to force the distal end of the outer bendable shaft 392 against the collar 462, the shaft is biased, by the notches 456, to bend in a pre-determined manner.

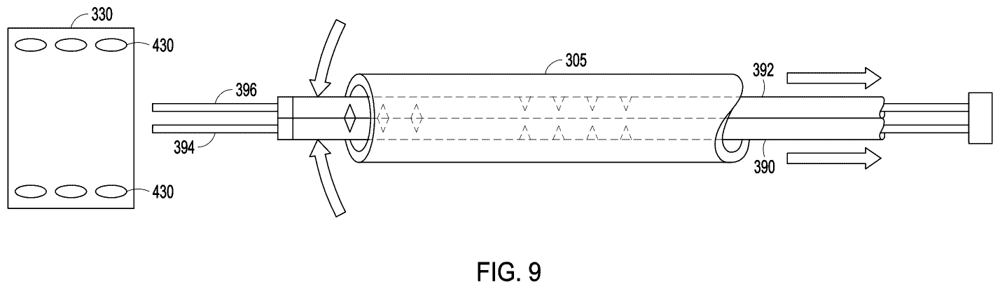

FIG. 9 shows the implant 330 and the implant delivery device, in accordance with one embodiment. Referring also to FIG. 4, here, the sheet-like implant 330 can include outer loops 430 at each side defined by one or more hemmed connections 431, which can be stitches or ultrasound welds, for example. The outer loops 430 are configured to connect with the first and second inner implant holding rods 394, 396, and after being implanted, the first and second inner implant holding rods 394, 396 are retracted through the loops 430, leaving the sheet-like implant 330 in place. Likewise, it is noted that when the force is removed on each end of the first and second bendable shafts 390, 392 by moving the actuator 342, 345 in the other direction, the first and second bendable shafts 390, 392 straighten out.

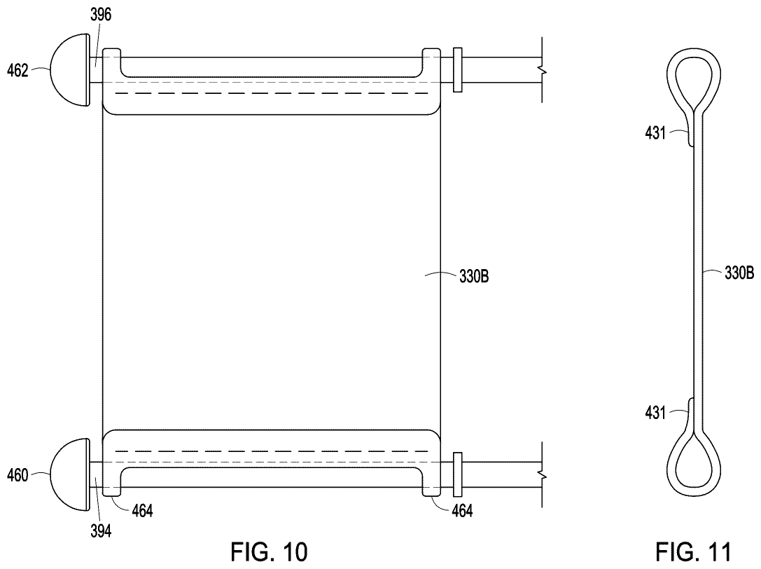

FIG. 10 shows a top view of an implant 330B, in accordance with one embodiment; And FIG. 11 shows a side view of the implant 330B, in accordance with one embodiment. In this example, the sheet-like implant 330B is configured such that, after being implanted, the first and second outer bendable shafts 390, 392 and the first and second inner implant holding rods 394, 396 are retracted and sacrificial portions 464 of the sheet-like implant 330B are torn way by the first and second inner implant holding rods 394, 396, leaving the major portion of the sheet-like implant 330B in place.

In this example, the sacrificial portions of the sheet-like implant 330B can include tear-away portions 464 of outer hemmed loops 430 on each side of the sheet-like implant 330B. Each of the first and second inner implant holding rods 394, 396 includes an expanded tip portion 466, 468 to engage the outer hemmed loops 430 as the first and second inner implant holding rods 394, 396 are retracted so as to tear away portions 464 of the hemmed loops 430.

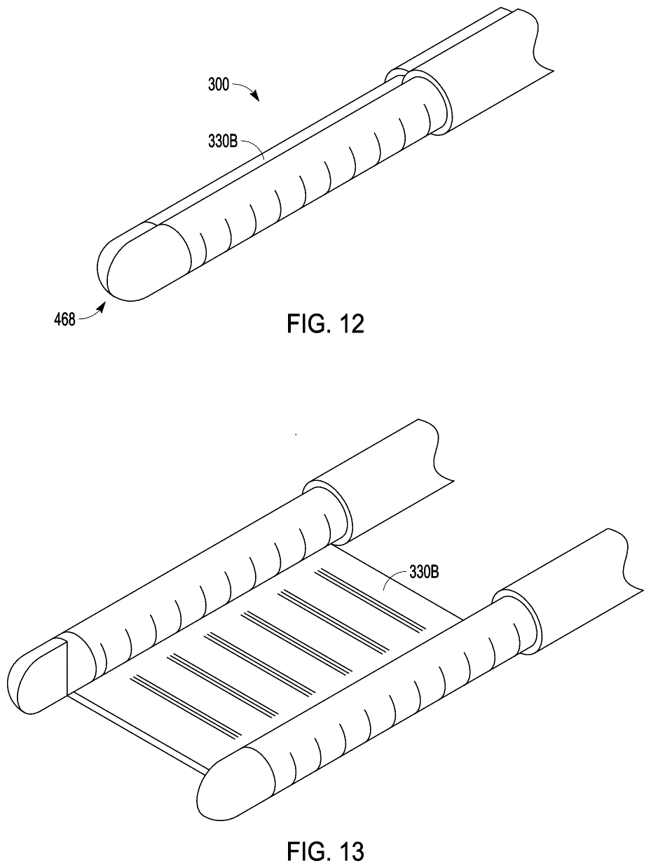

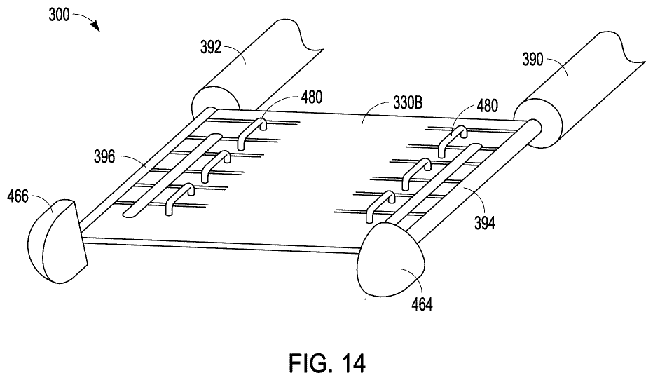

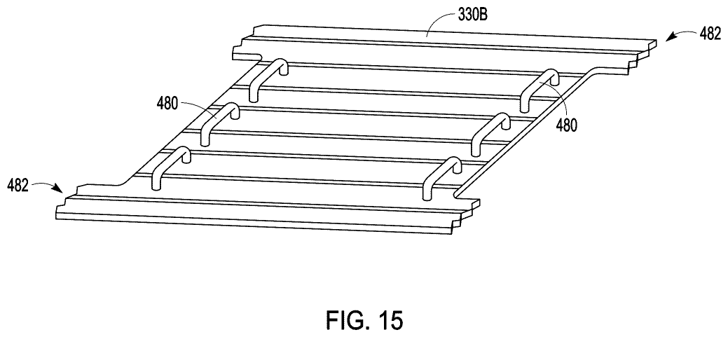

FIG. 12 shows a perspective view of the implant 330B and the delivery device, in accordance with one embodiment; FIG. 13 shows a perspective view of the implant 330B; FIG. 14 shows a perspective view of the implant 330B and the delivery device; and FIG. 15 shows a perspective view of the implanted implant 330B. Here, the expanded tip portions 466, 468 of each of the first and second inner implant holding rods 394, 396 include a half-dome shaped member, and the half-dome members mate to form a full-dome shape 470 when the first and second inner implant holding rods are together. After the implant 330B is unfurled by the spreading apart of shafts 390, 392, staples 480 can be inserted through the implant 330B to affix the implant 330B. When the delivery device 300 is removed, the domes of the expanded tip portions 466 and 468 tear away the sacrificial portions of the implant 330B at tear-away portions 382 leaving the major portion of the implant 330B in place.

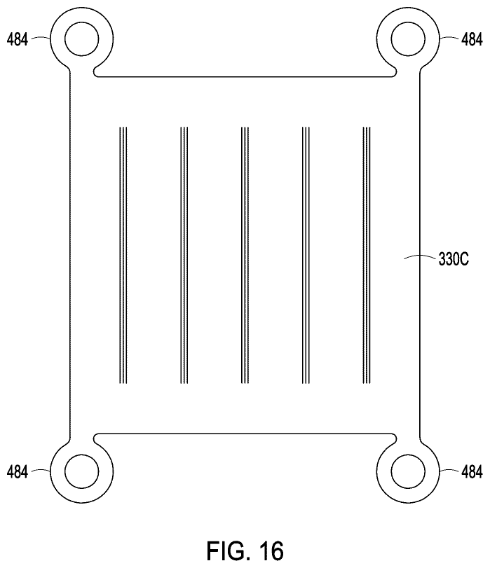

FIG. 16 shows a top view of an implant 330C, in accordance with one embodiment. Here, the implant 330C can include sacrificial portions including tear away eyelets 484 at each corner of the sheet-like implant 330C.

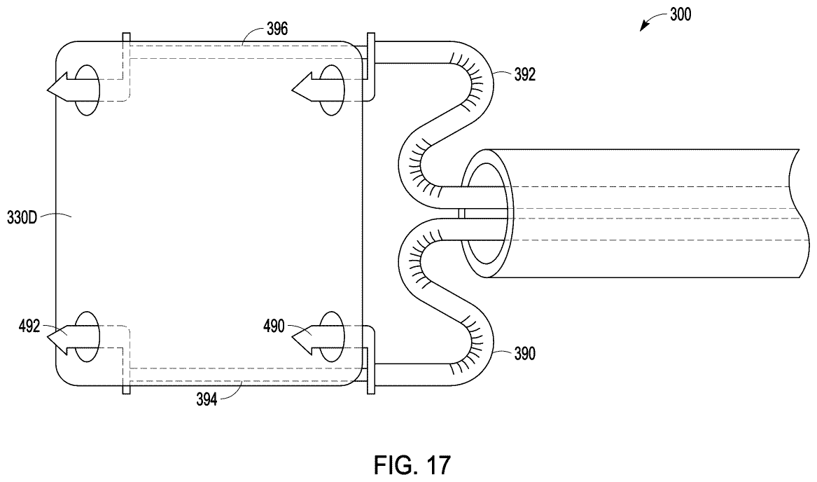

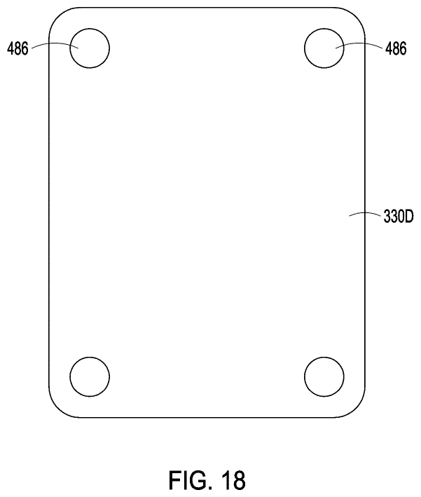

FIG. 17 shows a top view of an implant 330D and the delivery device 300, in accordance with one embodiment; and FIG. 18 shows a top view of the implant, in accordance with one embodiment.

Here, the sacrificial portions of the sheet-like implant 330 can include a hole 486 located at each corner of the sheet-like implant 330 and the first and second inner implant holding rods 394, 396 each include a pair of hooks 490, 492 to engage the sheet-like implant at each of the holes 486. In some examples, the hooks 490, 492 can tear away the holes 486, leaving the implant in place. In other examples, the hooks can slip through the holes 486 without tearing. Also, in this embodiment, the outer bendable shafts 390, 392 can be configured to bend in a recurved manner. Here, the notches in the outer shafts 390, 392 can be formed such that the outer shafts 390, 392 define a recurved S-shape when bent.

In summary, the present system provides an implant delivery device for positioning and deploying a sheet-like implant including an implant holding portion proximate a distal end of the implant delivery device, the implant holding portion being configured to retain a sheet-like implant during implantation of the sheet-like implant, wherein the implant holding portion is configured to hold the sheet-like implant in a collapsed delivery configuration while the sheet-like implant is pushed through a cannula and to stretch the sheet-like implant at its outer edges until the sheet-like implant is in a spread-out deployment configuration. This facilitates placing the staples or other fastening devices quickly and correctly in the edges of the graft.

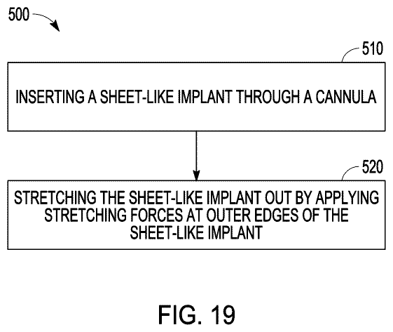

FIG. 19 shows a method for implanting a sheet-like implant, in accordance with one embodiment. The method (500) can include inserting a sheet-like implant through a cannula while the sheet-like implant is in a collapsed delivery configuration (510); and stretching the sheet-like implant out by applying stretching forces at outer edges of the sheet-like implant until the sheet-like implant is in a spread-out deployment configuration (520).

In further examples, stretching the sheet-like implant out includes attaching one side of the sheet-like implant to a first inner implant holding rod and attaching a second side of the sheet-like implant to a second inner implant holding rod; and bending the first and second inner implant holding rods away from each other.

In some examples, bending the first and second inner implant holding rods away from each other includes mounting each of the first and second inner implant holding rods within a first outer bendable shaft and a second outer bendable shaft, respectively, wherein a distal end of each of the first and second outer bendable shaft are configured to bend to convert the sheet-like implant from the collapsed delivery configuration to the spread-out deployment configuration.

The method of delivering the implant may include inserting a cannula into an incision in the skin of a patient. In some embodiments, the method may include placing a sheet-like implant into the implant holding portion of the implant delivery device. However, in other embodiments, this process of loading the implant into the implant delivery device may be done as a manufacturing step, and the loaded implant delivery device may be provided to the surgeon ready for use.

In addition, once the cannula is inserted, the method may also include arthroscopically inserting the implant holding portion of the implant delivery device through the cannula to a surgical site. It will be noted that, in some cases, the implant delivery device may be used in non-arthroscopic (e.g., open) surgeries. It will be further noted that the disclosed implant delivery device need not necessarily be used with a cannula such as cannula 305. If the implant delivery device were to be used during an open surgery, the cannula would not be necessary.

The method can further involve securing of the implant to tissue/bone at the surgical site, for example, with one or more anchors, such as staples. Once the implant has been secured, the outer shafts can be straightened and the instrument (i.e., the implant delivery device) may be removed from the surgical site.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An implant delivery device, comprising:
a handle disposed at a proximal end of the implant delivery device and configured to be grasped by a user;
a first outer bendable shaft extending from the handle to a distal end of the implant delivery device and a second outer bendable shaft extending from the handle to a distal end of the implant delivery device and positioned adjacent to the first outer bendable shaft; and
an implant holding portion proximate the distal end of the first and second outer bendable shafts, the implant holding portion being configured to retain a sheet-like implant during implantation of the sheet-like implant by being attached to outer edges of the sheet-like implant;
wherein a distal end of each of the first and second outer bendable shafts is configured to bend to convert the sheet-like implant from a collapsed delivery configuration to a spread-out deployment configuration.

2. The implant delivery device of claim 1, wherein the distal end of the first outer bendable shaft bends outwards from a longitudinal axis in one direction and the distal end of the second outer bendable shaft bends outwards from a longitudinal axis in a second, opposite direction.

3. The implant delivery device of claim 2, wherein the implant holding portion including a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant, and wherein as the distal ends of the first and second outer bendable shafts bend outwards, the sheet-like implant becomes stretched between the first inner implant holding rod and the second inner implant holding rod.

4. The implant delivery device of claim 1, wherein the distal end of each of the first and second outer bendable shafts includes a plurality of notches configured to bias the first and second bendable shafts to bend when a force is exerted on each end of the first and second bendable shafts.

5. The implant delivery device of claim 4, wherein the plurality of notches on each of the first and second outer bendable shafts includes a group of proximally located notches on an outer surface of each of the first and second outer bendable shafts and a group of distally located notches on an inner surface of each of the first and second outer bendable shafts.

6. The implant delivery device of claim 4, wherein the implant holding portion including a first inner implant holding rod located within the first outer bendable shaft and a second inner implant holding rod located within the second outer bendable shaft, and wherein the force is exerted on a proximal end of each of the first and second outer bendable shafts by an actuator within the handle and an opposite force is exerted on a distal end of each of the first and second outer bendable shafts by a collar on each of the first and second inner implant holding rods.

7. The implant delivery device of any of claim 6, wherein when the force is removed on each end of the first and second bendable shafts, the first and second bendable shafts straighten out.

8. The implant delivery device of claim 1, wherein the implant holding portion including a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant, and wherein the sheet-like implant includes outer loops at each side to connect with the first and second inner implant holding rods, and after being implanted, the first and second inner implant holding rods are retracted through the loops, leaving the sheet-like implant in place.

9. The implant delivery device of claim 1, wherein the implant holding portion including a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant, and wherein the sheet-like implant is configured such that, after being implanted, the first and second outer bendable shafts and first and second inner implant holding rods are retracted and sacrificial portions of the sheet-like implant are torn way by the first and second inner implant holding rods, leaving the sheet-like implant in place.

10. The implant delivery device of claim 9, wherein the sacrificial portions of the sheet-like implant include tear-away portions of outer hemmed loops on each side of the sheet-like implant.

11. The implant delivery device of claim 10, wherein each of the first and second inner implant holding rods includes an expanded tip portion to engage the outer hemmed loops as the first and second inner implant holding rods are retracted so as to tear away portions of the hemmed loops.

12. The implant delivery device of claim 11, wherein the expanded tip portion of each of the first and second inner implant holding rods includes a half-dome member, and the half-dome members mate to form a full-dome shape when the first and second inner implant holding rods are together.

13. The implant delivery device of claim 9, wherein the sacrificial portions of the sheet-like implant includes tear away eyelets at each corner of the sheet-like implant.

14. The implant delivery device of claim 9, wherein the sacrificial portions of the sheet-like implant includes a hole located at each corner of the sheet-like implant and the first and second inner implant holding rods each include a pair of hooks to engage the sheet-like implant at each of the holes.

15. An implant delivery device, comprising:
an implant holding portion proximate a distal end of the implant delivery device, the implant holding portion being configured to retain a sheet-like implant during implantation of the sheet-like implant, wherein the implant holding portion is configured to hold the sheet-like implant in a collapsed delivery configuration while the sheet-like implant is pushed through a cannula and to stretch the sheet-like implant at its outer edges until the sheet-like implant is in a spread-out deployment configuration.

16. The implant delivery device of claim 15, further including first and second outer bendable shaft which are configured to bend with the distal end of the first outer bendable shaft bending in one direction and the distal end of the second outer bendable shaft being bent in a second, opposite direction, wherein as the distal ends of the first and second outer bendable shafts are bent, the sheet-like implant becomes stretched, and wherein the implant holding portion includes a first inner implant holding rod located within the first outer bendable shaft and a second inner implant holding rod located within the second outer bendable shaft, wherein the first inner implant holding rod is configured to be removably attached to one side of the sheet-like implant and the second inner implant holding rod is configured to be removably attached to a second side of the sheet-like implant.

17. The implant delivery device of claim 16, wherein the distal end of each of the first and second outer bendable shafts includes a plurality of notches configured to bias the first and second bendable shafts to bend when a force is exerted on each end of the first and second bendable shafts.

18. A method for implanting a sheet-like implant, the method comprising:
inserting a sheet-like implant through a cannula while the sheet-like implant is in a collapsed delivery configuration; and
stretching the sheet-like implant out by applying stretching forces at outer edges of the sheet-like implant until the sheet-like implant is in a spread-out deployment configuration.

19. The method of claim 18, wherein stretching the sheet-like implant out includes attaching one side of the sheet-like implant to a first inner implant holding rod and attaching a second side of the sheet-like implant to a second inner implant holding rod; and bending the first and second inner implant holding rods away from each other.

20. The method of claim 19, wherein bending the first and second inner implant holding rods away from each other includes mounting each of the first and second inner implant holding rods within a first outer bendable shaft and a second outer bendable shaft, respectively, wherein a distal end of each of the first and second outer bendable shaft are configured to bend to convert the sheet-like implant from the collapsed delivery configuration to the spread-out deployment configuration.
